(19) United States
(12) Patent Application Publication
(10) Pub. No.: US 2024/0366637 A1
(43) Pub. Date: Nov. 7, 2024

(54) Compounds inhibiting the synergistic carsinogenic effect of heavy metals in the presence of other carcinogens for use in the treatment of cancer

(71) Applicant: Cancer Research and Biotechnology AG, Baar (CH)

(72) Inventor: Kari Sarvanto, Baar (CH)

(21) Appl. No.: 18/562,865

(22) PCT Filed: May 20, 2022
(86) PCT No.: PCT/EP2022/063811

§ 371 Date: Nov. 21, 2023

(30) Foreign Application Priority Data
May 21, 2021 (FI) 20215608

(51) Int. Cl. A61K 31/6615; A61K 31/185; A61K 33/30; C12Q 1/02

(52) U.S. Cl. CPC A61K 31/6615; A61K 31/185; A61K 33/30; C12Q 1/025

(57) ABSTRACT

The present invention is directed to a composition or combination comprising (i) a compound inhibiting the synergistic carcinogenic effect of cadmium in the presence of another carcinogen; and (ii) optionally a heavy metal chelating or binding compound; for use in the prevention or treatment of cancer in a patient or the prevention of the recurrence of cancer in a patient. The present invention is also directed to a method for detecting anticarcinogens comprising the steps of: providing living cells in a cell culture; culturing said cells in the presence of cadmium, a carcinogen and a candidate anticarcinogen; and detecting those candidate anticarcinogens as anticarcinogens which prevent or decrease the carcinogenic effect of the combination of cadmium and the carcinogen to said cells during the culturing step.

### FIELD OF THE INVENTION

The present invention relates generally to the field of molecular biology and medicine. In particular, the present invention relates to anticarcinogens useful for cancer treatments or preventing cancers or cancer recurrence.

### BACKGROUND OF THE INVENTION

In 1984, Mandel and Ryser disclosed that cadmium chloride (CdCl₂) increased the mutagenicity of two common nitrosoamines in synergistic fashion, at the level up to 30-fold greater than expected from simple additivity. The authors raised the possibility that cadmium and nitrosoamines may have synergistic effects as environmental carcinogens. In a subsequent study, the authors (Mandel and Ryser, 1987) disclosed the carcinogenic synergism of cadmium and N-methyl-N-nitrosourea (MNU) seemed to depend on the mutagenicity of MNU and not on the cytotoxicity of cadmium.

Harrison and Heath (1986) disclosed data on lung carcinogenesis in a mouse model showing that overall lung tumour incidence rate in the group of animals receiving crocidolite asbestos, cadmium and N-nitrosoheptamethyleneimine (NHMI) was significantly higher than in the groups of animals receiving either crocidolite asbestos and cadmium together or crocidolite asbestos and NHMI together.

U.S. Pat. No. 10,463,695 (Siren) discloses that the ratio of zinc and cadmium is of significance for error-free proliferation and differentiation of cells. The author also states that an understanding of the role of cadmium in the etiology of cancer offers possibilities to gain a better understanding of cancer. In U.S. Pat. No. 10,463,695, a specific method of treatment of cancer is disclosed, wherein the reduction of the amount of cadmium in a patient is performed by means of blood transfusion or extracorporeal dialysis.

WO2020159987 discloses a cancer treatment which is based on the observation that various combination therapies that include metal chelators may be particularly beneficial for the treatment of various cancers including, e.g., leukemias. In WO2020159987, one, two, or more chelators are administered to a mammalian subject with a cancer to selectively bind one or more metals, such as copper (Cu), arsenic (As), cesium (Cs), and/or lead (Pb) in the subject.

In light of the above-mentioned prior art, there is still a need in the art to discover novel anticarcinogens which act directly against cancer causing mechanisms. The present invention provides a new principle for discovering anticarcinogens as well as methods and compounds to prevent a cancer, to treat cancer or to prevent cancer recurrence in patients who have suffered from the disease.

### SUMMARY OF THE INVENTION

The present invention is based on the identification of compounds that prevent or inactivate pro-mutagen activity of heavy metals. Specifically, the present inventors have performed tests to find compounds which disrupt the synergistic mutagenicity previously observed for the combination of cadmium and N-Methyl-N-nitrosourea (MNU). The compounds found in the present invention lay a foundation for preventive, therapeutic and prophylactic cancer treatments based on anticarcinogenics.

Accordingly, the present invention provides a composition or combination comprising
(i) a compound inhibiting the synergistic carcinogenic effect of cadmium in the presence of another carcinogen; and
(ii) optionally a heavy metal chelating or binding compound;
for use in the prevention or treatment of cancer in a patient or the prevention of the recurrence of cancer in a patient.

In other related aspect, the present invention provides a method of preventing or treating cancer in a subject comprising administering to said subject:
(i) a compound inhibiting the synergistic carcinogenic effect of cadmium in the presence of other carcinogens; and
(ii) optionally a heavy metal chelating or binding compound.

In other related aspect, the present invention provides a method for detecting anticarcinogens comprising the steps of:
providing living cells in a cell culture;
culturing said cells in the presence of cadmium, a carcinogen and a candidate anticarcinogen; and
detecting those candidate anticarcinogens as anticarcinogens which prevent or decrease the carcinogenic effect of the combination of cadmium and the carcinogen to said cells during the culturing step.

### BRIEF DESCRIPTION OF THE DRAWINGS

FIG. 1. Unithiol (i.e. DMPS) demonstrated a dose-dependent inhibition of mutagenicity-promoting synergy between cadmium and N-Methyl-N-nitrosourea. A control plate was grown without the presence of a candidate anticarcinogen.

FIG. 2. Phytic acid demonstrated a dose-dependent inhibition of mutagenicity-promoting synergy between cadmium and N-Methyl-N-nitrosourea.

### EMBODIMENTS

The present invention relates to approaches for treating cancer in a subject. In one embodiment of the invention, the subject is a human or a mammal, specifically a mammal or human patient, more specifically a human or a mammal suffering from cancer. In a specific embodiment of the invention, the subject is a human. A mammal may be selected from a group consisting of pets, domestic animals and production animals.

The approach can be used to treat any cancers or tumors, including both malignant and benign tumors, both primary tumors and metastases may be targets of the approach.

It is to be noted that when tobacco products, such as cigarettes and fermented dipping snuff, are used, these products give off several tobacco-specific nitrosamines (TSNAs), some of the most potent cancer-causing substances known. It is also known that tobacco products contain cadmium, e.g., it has been measured that each cigarette contains about 0.5-1.5 micrograms of cadmium. Without wishing to be bound by a theory, it is concluded herein that the compounds of the present invention and combinations thereof are able to decrease the co-carcinogenic effect of cadmium and TSNAs. Therefore, the present invention is particularly for use in the prevention or treatment of cancers related to constant use of tobacco products. In preferred embodiments, the present invention is for use in the prevention (particularly prevention of cancer recurrence) or treatment of lung cancers, stomach cancer, oral cavity cancers such as mouth cancers, pharynx cancers, oesophageal cancers and larynx cancers.

As used herein, the term “treatment” or “treating” refers to administration of at least the composition of the present invention to a subject, preferably a mammal or human subject, for purposes which include not only complete cure but also prophylaxis, amelioration, or alleviation of disorders or symptoms related to a cancer or tumor. Therapeutic effect may be assessed by monitoring the symptoms of a patient, tumor markers in blood, or for example a size of a tumor or the length of survival of the patient.

Before classifying a human or animal patient as suitable for the therapy of the present invention, the clinician may examine a patient. Based on the results deviating from the normal and revealing a tumor or cancer, the clinician may suggest treatment of the present invention for a patient. In an embodiment of the invention, the subject or patient has already failed at least one previous treatment of cancer such as chemotherapy or immunotherapy treatment, i.e. the cancer of the patient is a chemotherapy and/or immunotherapy refractory tumor. In a preferred embodiment, the subject or patient has a successfully treated cancer and the present composition is administered to said subject or patient in order to prevent cancer recurrence.

In addition to the active agent of the invention, the composition of the invention may also comprise other therapeutically effective agents, any other agents such as pharmaceutically acceptable carriers, buffers, excipients, adjuvants, additives, preservatives, antiseptics, filling, stabilising and/or thickening agents, and/or any components normally found in corresponding products. Selection of suitable ingredients and appropriate manufacturing methods for formulating the compositions belongs to general knowledge of a man skilled in the art.

The composition of the invention may be in any form, such as solid, semisolid or liquid form, suitable for administration. The therapeutic agents may be formulated into a composition in a free base, neutral or salt form, or in a complex form. Pharmaceutically acceptable salts formed with the free carboxyl groups can also be derived from inorganic bases.

A formulation can be selected from a group consisting of, but not limited to, solutions, emulsions, suspensions, tablets, pellets and capsules. The compositions of the current invention are not limited to a certain formulation, instead the composition can be formulated into any known pharmaceutically acceptable formulation. The pharmaceutical compositions may be produced by any conventional processes known in the art. The formulations are easily administered in a variety of dosage forms such as formulated for parenteral administrations such as injectable solutions, or aerosols for delivery to the lungs, or formulated for alimentary administrations such as drug release capsules and the like.

The formulations of the present disclosure can also be presented as functional food, a nutraceutical product or a food supplement. A food supplement (i.e. a dietary supplement) is a manufactured product intended to supplement one's diet by taking a pill, capsule, tablet, powder, or liquid. Supplements can also contain substances that have not been confirmed as being essential to life, but are marketed as having a beneficiary effect on the health condition, preferably inhibitory effects to emergence of certain diseases. A supplement can provide nutrients either extracted from food sources or that are synthetic in order to increase the quantity of their consumption. The class of common nutrient compounds in food supplements includes vitamins, minerals, fiber, fatty acids, and amino acids. A supplement can also contain additives, preservatives, filling, stabilising and/or thickening agents.

A pharmaceutical kit of the present invention comprises a composition of the present invention formulated in a first formulation and a zinc containing composition and/or a vitamin C containing composition is/are formulated in a second formulation. In another embodiment of the invention the first and the second formulations are for simultaneous or sequential, in any order, administration to a subject. In another embodiment, said kit is for use in the treatment of cancer or tumor.

Any conventional method may be used for administration of the composition to a subject. The route of administration depends on the formulation or form of the composition, the disease, location of tumors, the patient, comorbidities and other factors. Accordingly, the dose amount and dosing frequency of each therapeutic agent in the combination depends in part on the particular therapeutic agent, the severity of the cancer being treated, and patient characteristics. Preferably, a dosage regimen maximizes the amount of each therapeutic agent delivered to the patient consistent with an acceptable level of side effects.

The administration of the active compound of the invention is preferably conducted through an oral administration or an intratumoral, intra-arterial, intravenous, intrapleural, intravesicular, intracavitary or peritoneal injection. Any combination of administrations is also possible. The approach can give systemic efficacy despite local injection.

Any other treatment or combination of treatments may be used in addition to the therapies of the present invention. In a specific embodiment the method or use of the invention further comprises administration of concurrent or sequential radiotherapy, chemotherapy, antiangiogenic agents or targeted therapies, such as alkylating agents, nucleoside analogs, cytoskeleton modifiers, cytostatic agents, monoclonal antibodies, kinase inhibitors or other anti-cancer drugs or interventions (including surgery) to a subject.

Accordingly, the present invention is directed to a composition or combination comprising
(i) a compound inhibiting the synergistic carcinogenic effect of cadmium in the presence of another carcinogen; and
(ii) optionally a heavy metal chelating or binding compound, preferably selected from a group consisting of: EDTA, dimercaptosuccinic acid (DMSA) and dimercaprol (BAL) or a salt thereof;
for use in the prevention or treatment of cancer in a patient or the prevention of the recurrence of cancer in a patient.

In a preferred embodiment said composition or combination further comprises zinc and preferably vitamin C. Preferably, zinc is in said composition or combination in the form of chelated zinc such as zinc gluconate, zinc glycinate, zinc acetate, zinc sulfate, zinc picolinate, zinc orotate, zinc citrate or a mixture thereof.

In another preferred embodiment, said patient is treated for cancer, said patient has been treated for cancer and/or said patient has recovered from a cancer.

In preferred embodiments, said compound inhibiting the synergistic carcinogenic effect of cadmium is Unithiol (2,3-Dimercapto-1-propanesulfonic acid, DMPS) or a pharmaceutically acceptable salt thereof such as sodium 2,3-Dimercapto-1-propanesulfonate. In a preferred embodiment, DMPS or a salt thereof can be orally administered at a dose of about 200-400 mg daily, depending on the clinical context.

In other preferred embodiments, said compound inhibiting the synergistic carcinogenic effect of cadmium is phytic acid, also known as inositol hexaphosphate (IP6), or a pharmaceutically acceptable salt thereof. The phytic acid or pharmaceutically acceptable phytate salt may be absorbed into or adsorbed onto a solid carrier to facilitate pharmaceutical administration. For example, phytic acid may be formulated into a starch powder by spray drying or vacuum drying an aqueous mixture of phytic acid and dextrin. The preferred composition for administration in oral dosage form is the monopotassium phytate salt, which may be prepared from commercially and readily available sodium phytate.

In another preferred embodiment, said composition comprises both above-mentioned active agents, i.e. a mixture of DMPS and phytic acid compounds as disclosed in the present invention.

In a preferred embodiment. said compound inhibiting the synergistic carcinogenic effect of cadmium is obtained by providing a cell based screening system for detection of mutagens and contacting a candidate compound with said screening system in the presence of cadmium and at least one other carcinogen such as a nitrosamine, wherein said candidate compound is an inhibitor of the synergistic carcinogenic effect of cadmium if the level of mutagenesis in the cells is lower in the presence of said candidate compound than in the absence of said candidate compound.

In a preferred embodiment, said cell based screening system for detection of mutagenes is the Ames test.

In another preferred embodiment, i) said compound inhibiting the synergistic carcinogenic effect of cadmium, ii) said heavy metal chelating or binding compound; iii) and optional further compounds such as zinc are administered simultaneously or sequentially, in any order, to a patient.

In another aspect, the present invention is directed to a method for detecting anticarcinogens comprising the steps of:
providing living cells in a cell culture;
culturing said cells in the presence of cadmium, a carcinogen and a candidate anticarcinogen; and
detecting those candidate anticarcinogens as anticarcinogens which prevent or decrease the carcinogenic effect of the combination of cadmium and the carcinogen to said cells during the culturing step. In other words, said candidate anticarcinogen is an inhibitor of the synergistic carcinogenic effect of cadmium if the level of mutagenesis in the cells is lower in the presence of said candidate anticarcinogen than in the absence of said candidate compound

In a preferred embodiment, said carcinogen is a nitrosamine, preferably N-Methyl-N-nitrosurea.

In another preferred embodiment, said carcinogen is selected from a group consisting of 4-Nitroquinoline-N-Oxide, 2-Nitrofluorene and 2-Aminoanthracene.

In another preferred embodiment, the anticarcinogen detected in the method is administered to a patient suffering from a cancer or to a subject in order to prevent a cancer or recurrence of cancer in said subject.

In another preferred embodiment, said method is the Salmonella typhimurium test (AMES test) for evaluating mutagenic properties of chemicals in vitro developed by Bruce N. Ames (Ames et al., 1973).

While the following examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, that is, a singular form, throughout this document does not exclude a plurality.

Reference throughout this specification to one embodiment or an embodiment means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

### EXPERIMENTAL SECTION

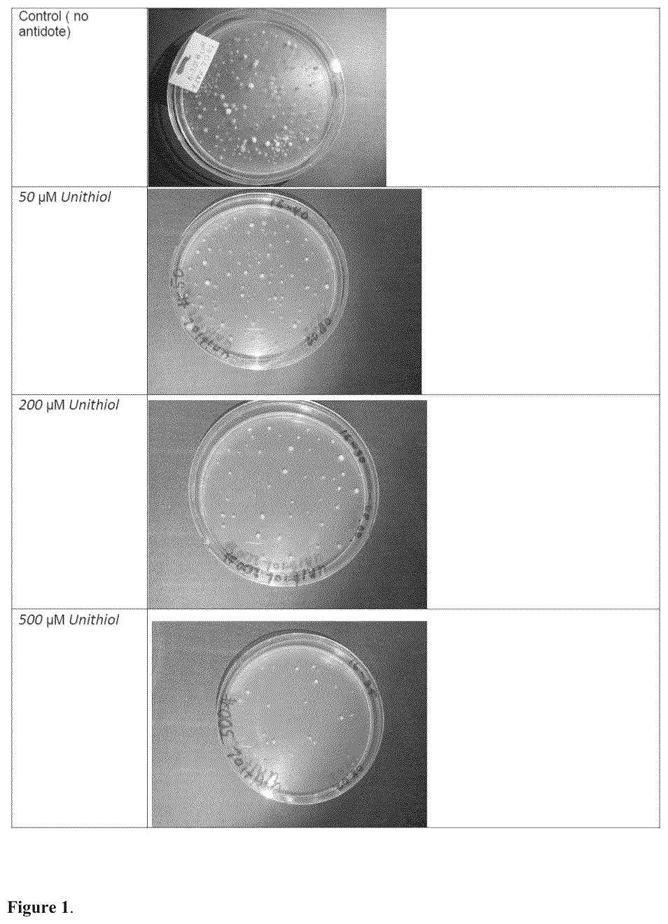

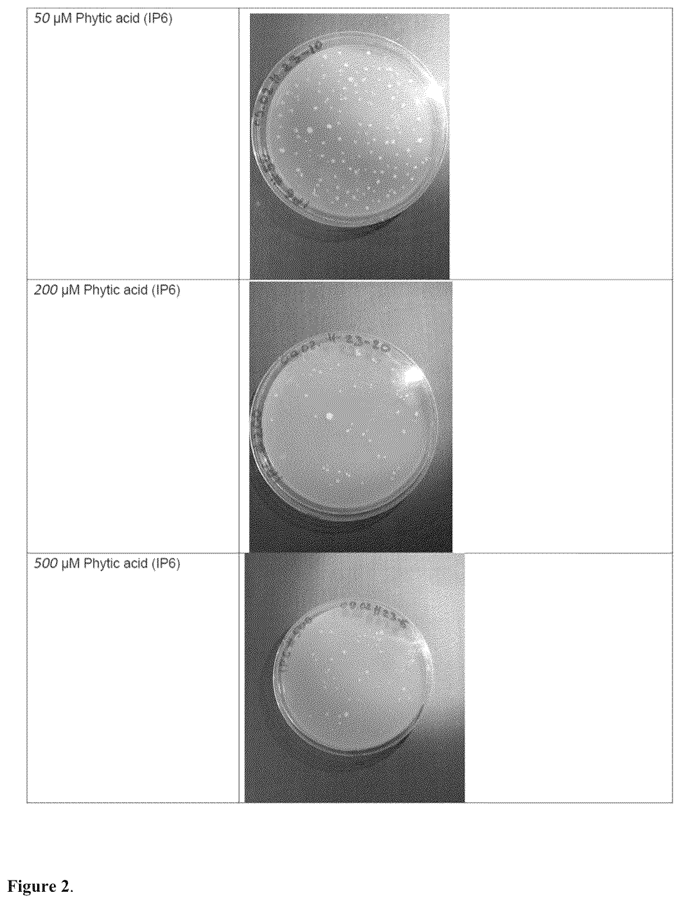

The operating hypothesis was tested with an AMES test (as described e.g. by Mandel and Ryser, 1984) based platform using Salmonella typhimurium TA1535 (Standard Ames Tester Strain), cadmium chloride as pro-mutagen (0.5 mM final concentration during treatment), N-Methyl-N-nitrosourea as mutagen (180 μM final concentration during treatment) and EDTA, phytic acid (IP6) and Unithiol (i.e. DMPS) as candidate cadmium-promoted mutagenicity antidotes (in various concentrations, see FIGS. 1 and 2).

The experiment included the following steps:
1. Healthy growth of bacterial cells
2. Absorption of cadmium chloride (as a non-genotoxic metabolic poison) under conditions of reduced metabolism (bacteria chilled on ice)
3. Addition of candidate antidotes and metabolic activation (fresh growth medium, 37° C.)
4. Addition of N-methyl-N-nitrosourea under metabolically active conditions to fix mutations (fresh growth medium, 37° C.)
5. Screening for mutant revertants on solid media (standard, second generation Petri dish-based AMES test)

### Results

Of the three tested candidates, phytic acid and Unithiol demonstrated a similar, dose-dependent inhibition of mutagenicity-promoting synergy between cadmium and N-methyl-N-nitrosourea (see FIGS. 1 and 2). In contrast, EDTA treatment did not inhibit the observed synergy.

### CONCLUSIONS

1. Phytic acid and Unithiol are confirmed candidate antidotes that may lead to formulations that remove/inactivate cadmium from tissues as part of cancer preventive, prophylactic or therapeutic regimens.

2. The developed AMES test protocol represents a convenient and easy screening method to identify cadmium-promoted mutagenicity antidotes alone or in combination.

### REFERENCES

Ames B N, Lee F D, Durston W E. An improved bacterial test system for the detection and classification of mutagens and carcinogens. Proc Natl Acad Sci USA. 1973 March; 70 (3): 782-6.
Harrison P T C and Heath J C. Apparent synergy in lung carcinogenesis: interactions between N-nitrosoheptamethyleneimine, particulate cadmium and crocidolite asbestos fibres in rats. Carsinogenesis, 7 (11): 1903-1908, 1986.
Mandel R and Ryser H J-P. Mutagenicity of cadmium in Salmonella typhimurium and its synergism with two nitrosamines. Mutation Research, 138 (1984) 9-16.
Mandel R and Ryser H J-P. Mechanism of synergism in the mutagenicity of cadmium and N-methyl-N-nitrosurea in Salmonella typhimurium: the effect of pH. Mutation Research, 176 (1987) 1-10.

### CITED PATENT DOCUMENTS

U.S. Pat. No. 10,463,695
WO2020159987

1. A composition comprising:
a compound inhibiting the synergistic carcinogenic effect of cadmium in the presence of another carcinogen;
for the prevention or treatment of cancer in a patient or the prevention of the recurrence of cancer in a patient.

2. The composition according to claim 1, further comprising a zinc chelate or a zinc salt.

3. (canceled)

4. The composition according to claim 1, wherein said compound inhibiting the synergistic carcinogenic effect of cadmium is 2,3-Dimercapto-1-propanesulfonic acid (DMPS) or a pharmaceutically acceptable salt thereof.

5. The composition according to claim 1, wherein said compound inhibiting the synergistic carcinogenic effect of cadmium is phytic acid or a pharmaceutically acceptable salt thereof.

6. The composition according to claim 1,
wherein said compound inhibiting the synergistic carcinogenic effect of cadmium is obtained by providing a cell based screening system for detection of mutagens and contacting a candidate compound with said screening system in the presence of cadmium and at least one other carcinogen, and
wherein said candidate compound is an inhibitor of the synergistic carcinogenic effect of cadmium if the level of mutagenesis in the cells is lower in the presence of said candidate compound than in the absence of said candidate compound.

7. The composition according to claim 6, wherein said cell-based screening system for detection of mutagenes is an Ames test.

8. (canceled)

9. (canceled)

10. The composition according to claim 1, comprising wherein the composition comprises: i) 2,3-Dimercapto-1-propanesulfonic acid (DMPS) or a pharmaceutically acceptable salt thereof; and ii) phytic acid or a pharmaceutically acceptable salt thereof.

11. (canceled)

12. The composition according to claim 1, wherein the composition is presented in the form of a pharmaceutical composition, a functional food, a nutraceutical product, or a food supplement.

13. A method of preventing or treating cancer in a subject, the method comprising administering to a subject the composition according to claim 1.

14. The method according to claim 13, wherein zinc is further administered to the subject.

15. The method according to claim 13, wherein said subject is being simultaneously treated for cancer by another therapy, said patient has been treated for cancer, and/or said patient has recovered from cancer.

16. The method according to claim 13, wherein said compound inhibiting the synergistic carcinogenic effect of cadmium is 2,3-dimercapto-1-propanesulfonic acid (DMPS) or a pharmaceutically acceptable salt thereof.

17. The method according to claim 13, wherein said compound inhibiting the synergistic carcinogenic effect of cadmium is phytic acid or a pharmaceutically acceptable salt thereof.

18. The method according to claim 13, wherein said compound inhibiting the synergistic carcinogenic effect of cadmium is obtained by providing a cell-based screening system for detection of mutagens and contacting a candidate compound with said screening system in the presence of cadmium and at least one other carcinogen, and
wherein said candidate compound is an inhibitor of the synergistic carcinogenic effect of cadmium if the level of mutagenesis in the cells is lower in the presence of said candidate compound than in the absence of said candidate compound.

19. The method according to claim 18, wherein said cell-based screening system for detection of mutagenes is an Ames test.

20. The method according to claim 13, further comprising administering a heavy metal chelating or binding compound, wherein said compound inhibiting the synergistic carcinogenic effect of cadmium, and said heavy metal chelating or binding compound are administered simultaneously or sequentially, in any order, to the subject.

21. A method for detecting anticarcinogens comprising the steps of:
providing living cells in a cell culture;
culturing said cells in the presence of cadmium, a carcinogen, and a candidate anticarcinogen; and
detecting those candidate anticarcinogens as anticarcinogens which prevent or decrease the carcinogenic effect of the combination of cadmium and the carcinogen to said cells during the culturing step.

22. The method according to claim 21, wherein said carcinogen is a nitrosamine.

23. (canceled)

24. (canceled)

25. The method according to claim 21, wherein said method is an AMES test.

26. (canceled)

27. (canceled)

28. The method according to claim 13, wherein the combination of i) 2,3-Dimercapto-1-propanesulfonic acid (DMPS) or a pharmaceutically acceptable salt thereof, and ii) phytic acid or a pharmaceutically acceptable salt thereof is administered to the subject simultaneously or sequentially, in any order.
